(19) United States
(12) Patent Application Publication
(10) Pub. No.: US 2009/0075397 A1
(43) Pub. Date: Mar. 19, 2009

(54) Method for characterizing sugar-binding interactions of biomolecules

(75) Inventors: Chun-Hung Lin, Taipei (TW); Chuan-Fa Chang, Taipei (TW)

Correspondence Address: COHEN, PONTANI, LIEBERMAN & PAVANE LLP, 551 FIFTH AVENUE, SUITE 1210, NEW YORK, NY, 10176, US

(73) Assignee: Academia Sinica, Taipei (TW)

(21) Appl. No.: 12/283,469

(22) Filed: Sep. 12, 2008

Related U.S. Application Data
(60) Provisional application No. 60/993,627, filed on Sep. 13, 2007.

(51) Int. Cl. G01N 33/543

(52) U.S. Cl. 436/531

(57) ABSTRACT

This invention provides a donor bead for use in an assay, wherein the bead (a) is coated with a layer of hydrogel having directly or indirectly bound thereto a polyacrylamide-supported sugar or a polyacrylamide-supported glycan, and (b) comprises a photosensitizer which, upon excitation by laser light of a suitable wavelength, converts ambient oxygen to singlet state oxygen. This invention also provides an acceptor bead for use in an assay, wherein the bead (a) is coated with a layer of hydrogel having directly or indirectly bound thereto a polyacrylamide-supported sugar or a polyacrylamide-supported glycan, and (b) comprises a chemiluminescer and a fluorophore, whereby when the bead is contacted with singlet state oxygen, the singlet state oxygen reacts with the chemiluminescer which in turn activates the fluorophore so as to cause the emission of light of a predetermined wavelength. This invention further provides related kits, detection methods and characterization methods.

### RELATED APPLICATIONS

This application claims priority from U.S. Provisional Patent Application Ser. No. 60/993,627 which was filed on Sep. 13, 2007, the contents of which are incorporated herein by reference.

### BACKGROUND OF THE INVENTION

1. Field of the Invention

Throughout this application, various publications are cited. The disclosure of these publications is hereby incorporated by reference into this application to describe more fully the state of the art to which this invention pertains.

2. Description of the Related Art

In eukaryotic cells, more than half of all proteins are glycosylated. Glycans expressed on the cell surface participate in many important cellular events through interactions with their corresponding proteins or receptors. Alterations in carbohydrate compositions are known to correlate with the changes in protein stability and clearance, as well as various important biological functions including cell-cell adhesion, inflammation, tumor metastasis, and infection of bacteria and viruses.

Although glycosylation plays a crucial role in the formation and progression of various diseases, the study of this subject is hampered by the structural heterogeneity and/or complexity of carbohydrates and the lack of effective tools available to date. As such, a number of techniques have been thus developed to characterize carbohydrate/protein interactions. The lectin blotting/binding assay has become a routine method to determine the interacting glyco-epitopes of glycoconjugates, but it has relatively low sensitivity and requires multiple time-consuming wash steps. The method based on surface plasmon resonance or quartz crystal microbalence monitors the interactions in real time in a quantitative manner by fixing proteins or functionalized carbohydrates to the surface of sensor chips. The sensitivity is, however, relatively low with low molecular weight carbohydrates, though the problem can be overcome by labeling sugars with organoplatinum(II). Recently carbohydrate microarrays have been developed to probe the carbohydrate binding properties of proteins or cells. Several chemical reactions have been utilized to immobilize specific carbohydrates to a solid support. For instance, a number of glycolipids and oligosaccharides with C14 hydrocarbon chains attached to the reducing end have been arranged in microplates through hydrophobic attachment. Using 1,3-dipolar cycloadditions, oligosaccharides were covalently immobilized on glass surface. Fluorous-tagged carbohydrates have been non-covalently bound to fluorinated glass. A high-content glycan array has been developed by applying a robotic microarray printing technology to couple amine functionalized glycans to a glass slide containing succinimide esters. Hydrogel glycan microarrays were developed to detect the binding of neoglycoconjugates with proteins. The lectin-based frontal affinity chromatography (FAC) was developed to combine the advantages of mass spectrometric analysis and the highly specific binding nature of lectins. Based on the evanescent-field fluorescence-detection principle, the lectin microarray has been developed for rapid profiling of glycan patterns. Other fluorescence-based techniques, such as fluorescence polarization and two-photon fluorescence correlation, were applied to study some lectin-sugar interactions. Self-assembled monolayers that present carbohydrates have been shown to identify enzyme and protein binding activity by mass spectrometric characterization. Prepared by either a non-covalent attachment or immobilization with a covalent oxime bond, glycosaminoglycan microarrays have been shown their sulfation patterns encoding molecular recognition and activity.

### SUMMARY OF THE INVENTION

This invention provides a donor bead for use in an assay, wherein the bead (a) is coated with a layer of hydrogel having directly or indirectly bound thereto a polyacrylamide-supported sugar or a polyacrylamide-supported glycan, and (b) comprises a photosensitizer which, upon excitation by laser light of a suitable wavelength, converts ambient oxygen to singlet state oxygen.

This invention also provides an acceptor bead for use in an assay, wherein the bead (a) is coated with a layer of hydrogel having directly or indirectly bound thereto a polyacrylamide-supported sugar or a polyacrylamide-supported glycan, and (b) comprises a chemiluminescer and a fluorophore, whereby when the bead is contacted with singlet state oxygen, the singlet state oxygen reacts with the chemiluminescer which in turn activates the fluorophore so as to cause the emission of light of a predetermined wavelength.

This invention also provides a kit comprising, in separate compartments, (a) a donor bead (i) coated with a layer of hydrogel and (ii) comprising a photosensitizer which, upon excitation by laser light of a suitable wavelength, converts ambient oxygen to singlet state oxygen, and (b) (i) a polyacrylamide-supported sugar or a polyacrylamide-supported glycan, or (ii) reagents for making a polyacrylamide-supported sugar or a polyacrylamide-supported glycan.

This invention also provides a kit comprising, in separate compartments, (a) an acceptor bead (i) coated with a layer of hydrogel and (ii) comprising a chemiluminescer and a fluorophore, whereby when the bead is contacted with singlet state oxygen, the singlet state oxygen reacts with the chemiluminescer which in turn activates the fluorophore so as to cause the emission of light of a predetermined wavelength, and (b) (i) a polyacrylamide-supported sugar or a polyacrylamide-supported glycan, or (ii) reagents for making a polyacrylamide-supported sugar or a polyacrylamide-supported glycan.

This invention also provides a method for determining whether a lectin or antibody binds to a sugar or glycan comprising (a) contacting, under binding-permitting conditions, (i) the donor bead of claim 1 having bound to its surface the sugar or glycan in polyacrylamide-supported form, and (ii) an acceptor bead having the lectin or antibody bound to its surface, wherein the acceptor bead comprises a chemiluminescer and a fluorophore, whereby when the acceptor bead is contacted with singlet state oxygen, the singlet state oxygen reacts with the chemiluminescer which in turn activates the fluorophore so as to cause the emission of light of a predetermined wavelength, (b) exposing the resulting beads to laser light of a wavelength which excites the photosensitizer in the donor bead, and (c) determining whether light is emitted by the fluorophore in the acceptor bead, the emission of light indicating that the lectin or antibody binds to the sugar or glycan.

This invention also provides a method for determining whether a lectin or antibody binds to a sugar or glycan comprising (a) contacting, under binding-permitting conditions, (i) a donor bead having the lectin or antibody bound to its surface, and (ii) the acceptor bead of claim 4 having bound to its surface the sugar in polyacrylamide-supported form or the glycan in polyacrylamide-supported form, wherein the donor bead comprises a photosensitizer which, upon excitation by laser light of a suitable wavelength, converts ambient oxygen to singlet state oxygen, (b) exposing the resulting beads to laser light of a wavelength which excites the photosensitizer in the donor bead, and (c) determining whether light is emitted by the fluorophore in the acceptor bead, the emission of light indicating that the lectin or antibody binds to the sugar or glycan.

This invention also provides a method for characterizing a glycan with respect to the makeup of its sugar moieties comprising (a) contacting, under binding-permitting conditions, (i) donor beads of claim 1, each having the glycan bound to its surface in polyacrylamide-supported form, and (ii) a plurality of acceptor beads, each having bound to its surface a lectin or antibody recognizing a predetermined sugar moiety, wherein the acceptor bead comprises a chemiluminescer and a fluorophore, whereby when the acceptor bead is contacted with singlet state oxygen, the singlet state oxygen reacts with the chemiluminescer which in turn activates the fluorophore so as to cause the emission of light of a predetermined wavelength, and wherein acceptor beads having a lectin or antibody recognizing a predetermined sugar moiety are contacted with the donor beads in a compartment separate from those in which donor beads are contacted with acceptor beads having lectins or antibodies recognizing other predetermined sugar moieties, (b) exposing the resulting beads in each compartment to laser light of a wavelength which excites the photosensitizer in the donor beads, and (c) for each compartment, determining whether light is emitted by the fluorophore in the respective acceptor beads and thus whether the lectin or antibody is bound to its respective sugar moiety on the glycan, thereby characterizing the glycan.

Finally, this invention provides a method for characterizing a glycan with respect to the makeup of its sugar moieties comprising (a) contacting, under binding-permitting conditions, (i) acceptor beads of claim 4, each having the glycan bound to its surface in polyacrylamide-supported form, and (ii) a plurality of donor beads, each having bound to its surface a lectin or antibody recognizing a predetermined sugar moiety, wherein each donor bead comprises a photosensitizer which, upon excitation by laser light of a suitable wavelength, converts ambient oxygen to singlet state oxygen, and wherein donor beads having a lectin or antibody recognizing a predetermined sugar moiety are contacted with the acceptor beads in a compartment separate from those in which acceptor beads are contacted with donor beads having lectins or antibodies recognizing other predetermined sugar moieties, (b) exposing the resulting beads in each compartment to laser light of a wavelength which excites the photosensitizer in the donor beads, and (c) for each compartment, determining whether light is emitted by the fluorophore in the respective acceptor beads and thus whether the lectin or antibody is bound to its respective sugar moiety on the glycan, thereby characterizing the glycan.

### BRIEF DESCRIPTION OF THE DRAWINGS

FIG. 1. Schematic representation of the sugar/protein binding assay (A) Use of biotinylated sugars did not generate any signal in the binding assay using streptavidin-conjugated beads. (B) Positive signals were generated when biotinylated sugars were replaced with PAA-linked sugars.

FIG. 2. Sugar binding specificities of eleven lectins were indicated by relative intensities (y axis). The sugar identities are designated by numbers (x axis). Each of the results was averaged from at least three independent assays.

FIG. 3. Glyco-pattern analysis of eleven lectins which were profiled by six biotinylated glycoproteins. The binding specificities were indicated by relative intensities (y axis). The sugar identities are designated by colors (x axis). Each of the results was averaged from at least three independent assays.

FIG. 4. Receptor binding specificities of four recombinant hemagglutinins (HAs) as indicated by relative intensities (y axis). (A) H1N1(A/Beijing/262/95), (B) H5N1 (A/Vietnam/1203/04), (C) H3N2 (A/Wyoming/3/2003) and (D) H9N3 (A/HongKong/1073/99).

FIG. 5. Receptor binding specificities of four influenza virus strains as indicated by relative intensities (y axis). (A) H1N1 (A/Beijing/262/95), (B) H3N2 (A/Panama/2007/99), (C) H1N1 (A/Taiwan/1/86) and (D) H3N2 (A/Shangdong/9/93).

FIG. 6 (also called Scheme 1 (SI)). Preparation of biotinylated PAA-L-fucose 10 (glycan No. 6 in Table 1) and biotinylated PAA-lactose 11 (glycan No. 12 in Table 1).

FIG. 7 (SI). Relative intensities (indicated with bars) of seven antibodies with 30 PAA-sugars were determined with reference to the highest absorbance unit. The measurement was described in Experimental Section.

FIG. 8 (SI): Characterization of compound 2's molecular weight by gel filtration chromatography using HPLC. The inset plot shows the molecular weight calibration curve that was determined by using various molecular weight standards of polyacrylic acids.

FIG. 9 (SI): Concentration-dependent signals of the binding interaction between PAA-Le^{x} and anti-Le^{x} antibodies. When the concentration of PM-Le^{x} is too high, the resulting signal decreases due to “hook effect”, i.e. high concentration of PAA-Le^{x} results in the binding with donor beads, and the binding with acceptor beads as well. The outcome thus prevents both beads from binding to each other in solution.

FIG. 10A-10D shows data regarding compound 4 (shown in Scheme 1) (10A-10B) and compound 8 (10C-10D).

### DETAILED DESCRIPTION OF THE PRESENTLY PREFERRED EMBODIMENTS

This invention provides a donor bead for use in an assay, wherein the bead (a) is coated with a layer of hydrogel having directly or indirectly bound thereto a polyacrylamide-supported sugar or a polyacrylamide-supported glycan, and (b) comprises a photosensitizer which, upon excitation by laser light of a suitable wavelength, converts ambient oxygen to singlet state oxygen.

In one embodiment, (a) the bead is coated with streptavidin and the polyacrylamide-supported sugar or polyacrylamide-supported glycan is bound to the bead via a biotin/streptavidin link, and (b) the photosensitizer is pthalocyanine. In another embodiment, the bead has a polyacrylamide-supported glycan bound thereto.

This invention also provides an acceptor bead for use in an assay, wherein the bead (a) is coated with a layer of hydrogel having directly or indirectly bound thereto a polyacrylamide-supported sugar or a polyacrylamide-supported glycan, and (b) comprises a chemiluminescer and a fluorophore, whereby when the bead is contacted with singlet state oxygen, the singlet state oxygen reacts with the chemiluminescer which in turn activates the fluorophore so as to cause the emission of light of a predetermined wavelength.

In one embodiment, (a) the bead is coated with streptavidin and the polyacrylamide-supported sugar or a polyacrylamide-supported glycan is bound to the bead via a biotin/streptavidin link, (b) the chemiluminescer is a thioxene derivative which luminesces at a wavelength of 370 nm, and (c) the fluorophore shifts 370 nm luminescence to a wavelength of from 520 nm to 620 nm. In another embodiment, the bead has a polyacrylamide-supported glycan bound thereto.

This invention also provides a kit comprising, in separate compartments, (a) a donor bead (i) coated with a layer of hydrogel and (ii) comprising a photosensitizer which, upon excitation by laser light of a suitable wavelength, converts ambient oxygen to singlet state oxygen, and (b) (i) a polyacrylamide-supported sugar or a polyacrylamide-supported glycan, or (ii) reagents for making a polyacrylamide-supported sugar or a polyacrylamide-supported glycan.

In one embodiment, (a) the donor bead is further coated with streptavidin, (b) the polyacrylamide-supported sugar or polyacrylamide-supported glycan is conjugated with biotin, and (c) the photosensitizer is pthalocyanine. In another embodiment, the kit further comprises, in a separate compartment, an acceptor bead comprising a chemiluminescer and a fluorophore. In yet another embodiment, the chemiluminescer is a thioxene derivative which luminesces at a wavelength of 370 nm, and the fluorophore shifts 370 nm luminescence to a wavelength of from 520 nm to 620 nm.

This invention also provides a kit comprising, in separate compartments, (a) an acceptor bead (i) coated with a layer of hydrogel and (ii) comprising a chemiluminescer and a fluorophore, whereby when the bead is contacted with singlet state oxygen, the singlet state oxygen reacts with the chemiluminescer which in turn activates the fluorophore so as to cause the emission of light of a predetermined wavelength, and (b) (i) a polyacrylamide-supported sugar or a polyacrylamide-supported glycan, or (ii) reagents for making a polyacrylamide-supported sugar or a polyacrylamide-supported glycan.

In one embodiment, (a) the acceptor bead is further coated with streptavidin, (b) the polyacrylamide-supported sugar or polyacrylamide-supported glycan is conjugated with biotin, (c) the chemiluminescer is a thioxene derivative which luminesces at a wavelength of 370 nm, and (d) the fluorophore shifts 370 nm luminescence to a wavelength of from 520 nm to 620 nm. In another embodiment, the kit further comprises, in a separate compartment, a donor bead comprising a photosensitizer. In yet another embodiment, the photosensitizer is pthalocyanine.

This invention also provides a method for determining whether a lectin or antibody binds to a sugar or glycan comprising (a) contacting, under binding-permitting conditions, (i) the donor bead of claim 1 having bound to its surface the sugar or glycan in polyacrylamide-supported form, and (ii) an acceptor bead having the lectin or antibody bound to its surface, wherein the acceptor bead comprises a chemiluminescer and a fluorophore, whereby when the acceptor bead is contacted with singlet state oxygen, the singlet state oxygen reacts with the chemiluminescer which in turn activates the fluorophore so as to cause the emission of light of a predetermined wavelength, (b) exposing the resulting beads to laser light of a wavelength which excites the photosensitizer in the donor bead, and (c) determining whether light is emitted by the fluorophore in the acceptor bead, the emission of light indicating that the lectin or antibody binds to the sugar or glycan.

In one embodiment, (a) the donor bead is coated with streptavidin and the polyacrylamide-supported sugar or polyacrylamide-supported glycan is bound to the bead via a biotin/streptavidin link, (b) the photosensitizer is pthalocyanine, (c) the acceptor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm. In another embodiment, the donor bead has a polyacrylamide-supported glycan bound thereto. In yet another embodiment, the method is performed using an assay well plate.

This invention also provides a method for determining whether a lectin or antibody binds to a sugar or glycan comprising (a) contacting, under binding-permitting conditions, (i) a donor bead having the lectin or antibody bound to its surface, and (ii) the acceptor bead of claim 4 having bound to its surface the sugar in polyacrylamide-supported form or the glycan in polyacrylamide-supported form, wherein the donor bead comprises a photosensitizer which, upon excitation by laser light of a suitable wavelength, converts ambient oxygen to singlet state oxygen, (b) exposing the resulting beads to laser light of a wavelength which excites the photosensitizer in the donor bead, and (c) determining whether light is emitted by the fluorophore in the acceptor bead, the emission of light indicating that the lectin or antibody binds to the sugar or glycan.

In one embodiment, (a) the acceptor bead is coated with streptavidin and the polyacrylamide-supported sugar or polyacrylamide-supported glycan is bound to the bead via a biotin/streptavidin link, (b) the photosensitizer is pthalocyanine, (c) the donor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm. In another embodiment, the acceptor bead has a polyacrylamide-supported glycan bound thereto. In yet another embodiment, the method is performed using an assay well plate.

This invention also provides a method for characterizing a glycan with respect to the makeup of its sugar moieties comprising (a) contacting, under binding-permitting conditions, (i) donor beads of claim 1, each having the glycan bound to its surface in polyacrylamide-supported form, and (ii) a plurality of acceptor beads, each having bound to its surface a lectin or antibody recognizing a predetermined sugar moiety, wherein the acceptor bead comprises a chemiluminescer and a fluorophore, whereby when the acceptor bead is contacted with singlet state oxygen, the singlet state oxygen reacts with the chemiluminescer which in turn activates the fluorophore so as to cause the emission of light of a predetermined wavelength, and wherein acceptor beads having a lectin or antibody recognizing a predetermined sugar moiety are contacted with the donor beads in a compartment separate from those in which donor beads are contacted with acceptor beads having lectins or antibodies recognizing other predetermined sugar moieties, (b) exposing the resulting beads in each compartment to laser light of a wavelength which excites the photosensitizer in the donor beads, and (c) for each compartment, determining whether light is emitted by the fluorophore in the respective acceptor beads and thus whether the lectin or antibody is bound to its respective sugar moiety on the glycan, thereby characterizing the glycan.

In one embodiment, (a) the donor bead is coated with streptavidin and the polyacrylamide-supported glycan is bound to the bead via a biotin/streptavidin link, (b) the photosensitizer is pthalocyanine, (c) the acceptor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm. In another embodiment, the method is performed using an assay well plate.

Finally, this invention provides a method for characterizing a glycan with respect to the makeup of its sugar moieties comprising (a) contacting, under binding-permitting conditions, (i) acceptor beads of claim 4, each having the glycan bound to its surface in polyacrylamide-supported form, and (ii) a plurality of donor beads, each having bound to its surface a lectin or antibody recognizing a predetermined sugar moiety, wherein each donor bead comprises a photosensitizer which, upon excitation by laser light of a suitable wavelength, converts ambient oxygen to singlet state oxygen, and wherein donor beads having a lectin or antibody recognizing a predetermined sugar moiety are contacted with the acceptor beads in a compartment separate from those in which acceptor beads are contacted with donor beads having lectins or antibodies recognizing other predetermined sugar moieties, (b) exposing the resulting beads in each compartment to laser light of a wavelength which excites the photosensitizer in the donor beads, and (c) for each compartment, determining whether light is emitted by the fluorophore in the respective acceptor beads and thus whether the lectin or antibody is bound to its respective sugar moiety on the glycan, thereby characterizing the glycan.

In one embodiment, (a) the acceptor bead is coated with streptavidin and the polyacrylamide-supported glycan is bound to the bead via a biotin/streptavidin link, (b) the photosensitizer is pthalocyanine, (c) the donor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm. In another embodiment, the method is performed using an assay well plate.

### Experimental Details

### PART I

Synopsis

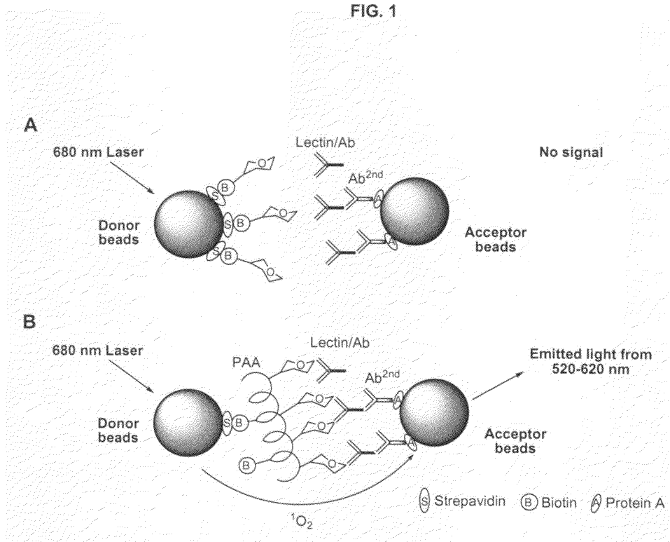

We report herein a high-throughput, homogenous and sensitive method to characterize protein-carbohydrate interactions and glyco-structures by in-solution proximity binding with photosensitizers. The technology, also called AlphaScreen™, was first described by Ullman et al. and has been used to study interactions between biomolecules. In these assays, a light signal is generated when a donor bead and an acceptor bead are brought into proximity. The donor beads contain phthalocyanine, a photosensitizer generating short-lived singlet oxygen upon irradiation at 680 nm. The singlet oxygen species diffuse only a short distance (˜200 nm) before decaying to the ground state. The acceptor beads contain a mixture of chemiluminescent molecules and fluorophores. When reacting with singlet oxygen, the chemiluminescent molecules undergo a series of chemical transformations that result in a time-delayed energy transfer to the fluorophores. The activated fluorophores, in turn, emit an amplified light signal at ˜600 nm, a shorter wavelength than the incident light. This cascade of reactions, coupled with time-resolved detection, results in a high signal with very low background. Streptavidin and protein A are coated on donor and acceptor beads for easy attachment of the molecules of interest (FIG. 1), respectively.

Results and Discussion

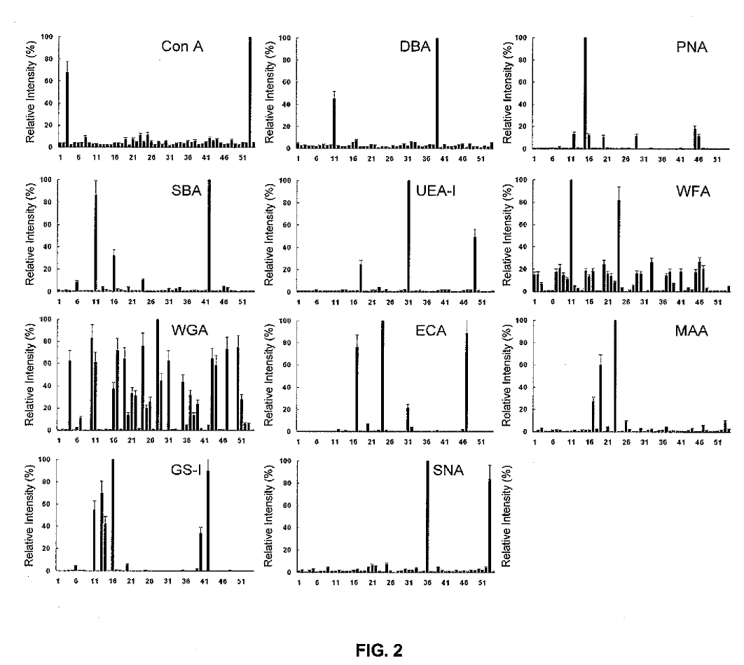

Our initial trial to study the binding interactions of biotinylated fucose and Lewis x (Lex) failed to produce any positive signal, i.e. both sugars linked to the donor beads did not bind with the lectin UEA-1 and anti-Lex antibody on the acceptor beads, respectively (FIG. 1A). Interestingly, the replacement with polyacrylamide (PAA)-supported fucose and Lex was able to produce signals, (FIG. 1B) which was realized due to the presence of multivalency. With this approach, more than 50 biotinylated PAA-sugars (Table 1) were then collected for further investigations. Some of them were synthesized according to known procedures (see Scheme 1 in supporting Information (SI)), while others were commercially available. Eighteen selected carbohydrate binding proteins, including eleven lectins (FIG. 2) and seven antibodies (see FIG. 1 (SI)), were profiled for their binding specificities. The signals were indicated with bars as relative intensities. The resulting lectin specificities were consistent with previously reported data. For instance, concanavalin A (Con A) binds preferentially to mannose and PNA binds to the Galb1-3GalNAc structure specifically. Likewise, each of the antibodies (e.g. anti-Lewis x and anti-sialyl Lewis a) was found to be highly specific for certain sugars (see FIG. 1 (SI)).

For successful conjugation on the acceptor beads, the antibody-bound proteins of interest must be recognized by protein A-containing acceptor beads. For instance, the proteins that contain a His-tag or FITC must use rabbit anti-His-tag or anti-FITC antibodies, respectively, as secondary antibodies for attachment to protein A-containing acceptor beads. This method usually provides good sensitivity with femtomole detection under optimized conditions that were dependent on the concentrations of PAA-sugars and protein analytes. Twenty ng/well PAA-sugars and 2-100 nM (equivalent to 2.5-125 ng/well) proteins were often required in a typical procedure.

TABLE 1
Saccharides immobilized on polyacrylamides.

| Glycan No. | Glycans |
|---|---|
| 1 | PAA-biotin |
| 2 | β-GlcNAc-spacei |
| 3 | α-Mannose |
| 4 | β-GlcNAc |
| 5 | β-GalNAc |
| 6 | α-L-Fuc |
| 7 | α-NeuAc |
| 8 | α-NeuGc |
| 9 | Glcα1-1Glc |
| 10 | GlcNAcβ1-1GlcNAc |
| 11 | GalNAcα1-3Gal |
| 12 | Galβ1-1Glc (Lactose) |
| 13 | Galα1-3Gal |
| 14 | Galα1-3GalNAc |
| 15 | Galβ1-3GalNAc |
| 16 | Galα1-1GlcNAc (α-LacNAc) |
| 17 | Galβ1-1GlcNAc (LacNAc) |
| 18 | Fucα1-2Gal |
| 19 | 3-HSO₃-Galβ1-4GlcNAc |
| 20 | Galβ1-3GlcNAc (Le^{c}) |
| 21 | NeuAcα2-6GalNAc |
| 22 | NeuGcα2-6GalNAc |
| 23 | 3-HSO₃-Galβ1-3GlcNAc |
| 24 | Galβ1-4(6-HSO₃)GlcNAc |
| 25 | 6-HSO₃-Galβ1-4GlcNAc |
| 26 | NeuAcα2-3Gal |
| 27 | NeuAcα2-3GalNAc |
| 28 | GlcNAcβ1-4GlcNAcβ1-4GlcNAc |
| 29 | GlcNAcβ1-3Galβ1-4GlcNAc |
| 30 | Fucα1-2Galβ1-3GlcNAc (H type1) |
| 31 | Fucα1-2Galβ1-4GlcNAc (H type2) |
| 32 | Galβ1-3(Fucα1-4)GlcNAc (Le^{a}) |
| 33 | Galβ1-4(Fucα1-3)GlcNAc (Le^{x}) |
| 34 | 3-HSO₃Galβ1-4(Fucα1-3)GlcNAc (HSO₃Le^{x} |
| 35 | NeuAcα2-3Galβ1-3GlcNAc |
| 36 | NeuAcα2-6Galβ1-4Glc |
| 37 | NeuAcα2-3Galβ1-4Glc |
| 38 | NeuAcα2-3(NeuAcα2-6)GalNAc |
| 39 | GalNAcα1-3(Fucα1-2)Gal (Type A) |
| 40 | Galα1-3(Fucα1-2)Gal (Type B) |
| 41 | 3-HSO₃Galβ1-3(Fucα1-4)GlcNAc (HSO₃Le^{a}) |
| 42 | Galα1-4Galβ1-4Glc |
| 43 | NeuAcα2-3Galβ1-4GlcNAc (Sialyl LacNAc) |
| 44 | NeuAcα2-3Galβ1-3GlcNAc (Sialyl Le^{c}) |
| 45 | Galβ1-3(NeuAcα2-6)GalNAc |
| 46 | Galβ1-3GlcNAcβ1-3Galβ1-4Glc |
| 47 | Galβ1-4GlcNAcβ1-3Galβ1-4Glc |
| 48 | Fucα1-2Galβ1-3(Fucα1-4)GlcNAc (Le^{b}) |
| 49 | Fucα1-2Galβ1-4(Fucα1-3)GlcNAc (Le^{y}) |
| 50 | NeuAcα2-3Galβ1-3(Fucα1-4)GlcNAc (Sialyl Le^{a}) |
| 51 | NeuAcα2-3Galβ1-4(Fucα1-3)GlcNAc (Sialyl Le^{x}) |
| 52 | (NeuAcα2-8)₅₋₆ |
| 53 | (NeuAcα2-6 Galβ1-4GlcNAcβ1-2Manα1-)₂- |
| | α3,6Manβ1-4GlcNAcβ1-4GlcNAc |
| 54 | H₂0 |

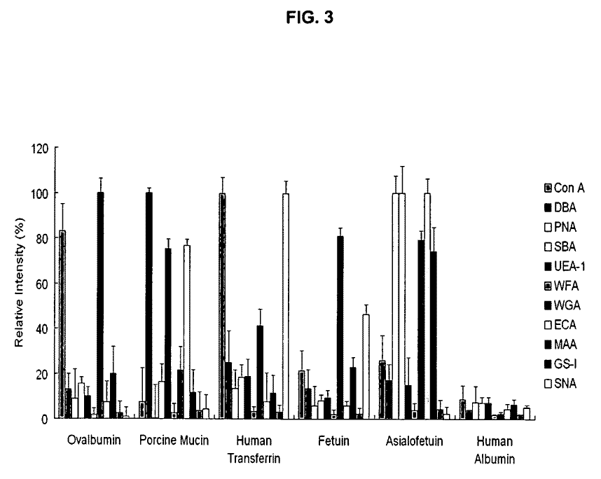

Moreover, the aforementioned lectins were used to characterize the glycan structures of six glycoproteins, such as ovalbumin, porcine mucin, human serum albumin, human transferrin, fetuin and asialofetuin. For immobilization on the donor beads, these proteins were biotinylated prior to the study (10-500 ng/well). Distinctive glycopattens were generated in accordance with the analysis of eleven lectins (FIG. 3). The results were similar to those obtained from lectin microarray or dot blot analysis.[26] Porcine mucin, for instance, was known to have fucosylated O-linked glycans with terminal residues of GalNAc, GlcNAc and NeuAc. Our analysis produced positive signals in the tests with the ECA, DBA, UEA-1 and WGA lectins, revealing that the mucin contains the determinants of Galb1-4GlcNAc, GalNAc, Fuca1-2Gal and GlcNAc/NeuAc, respectively. Man1-3(Fuca1-6)GlcNAc2- and NeuAca2-6Galb1-4GlcNAc-containing biantennary or triantennary N-glycans have been suggested as the major glycoforms of human transferrin.[26] Strong Con A and SNA signals were shown in our binding assay, but the relative intensity of GlcNAc-binding lectins was not as good as those of Con A and SNA, which was attributed to the interference by the terminal sialic acids.[27]

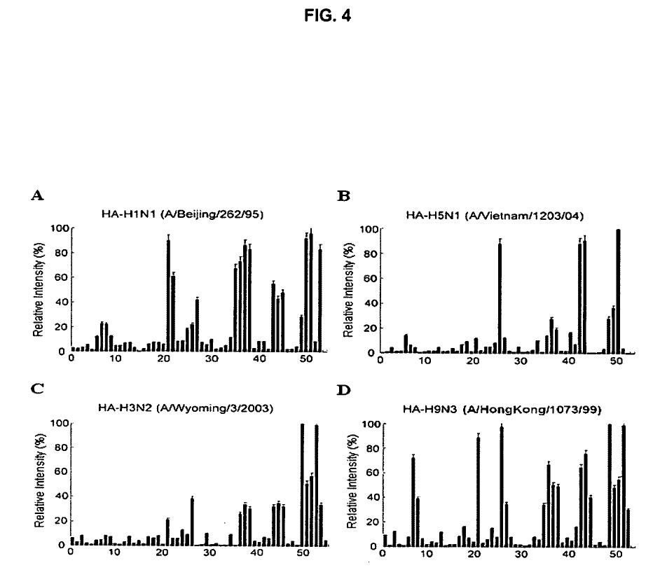

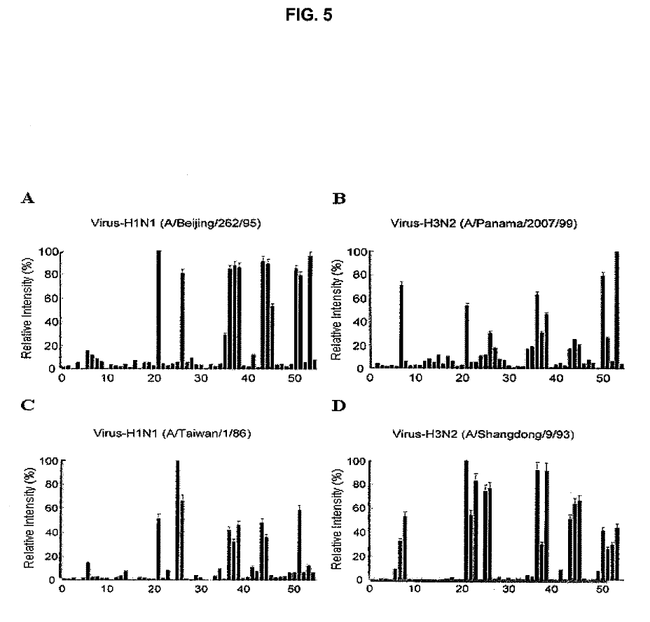

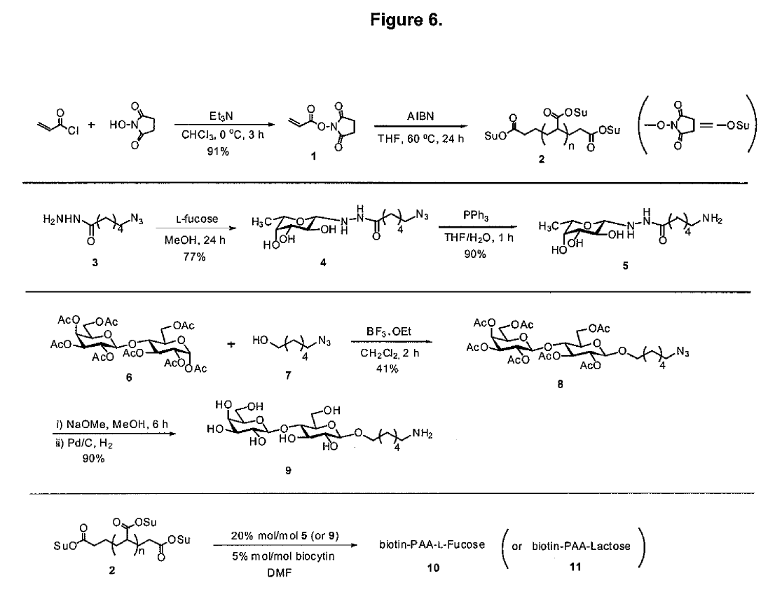

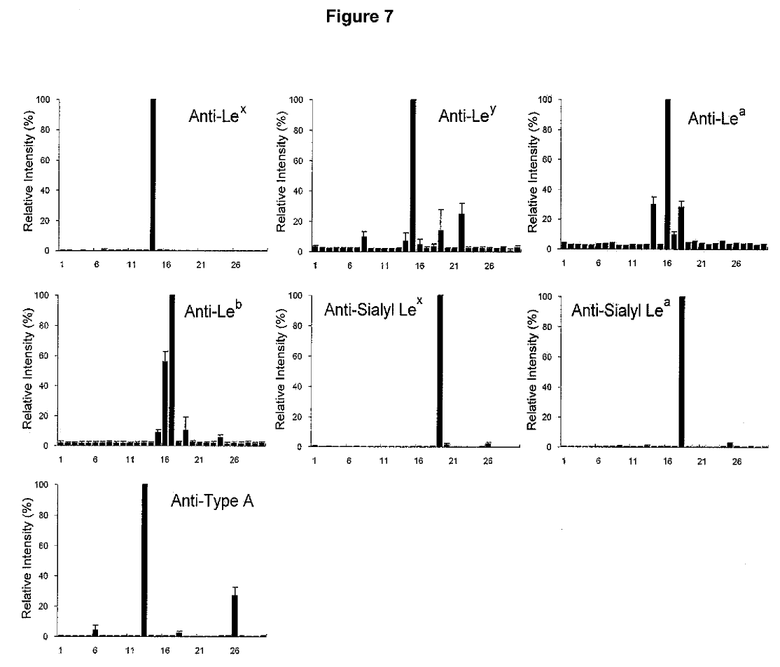

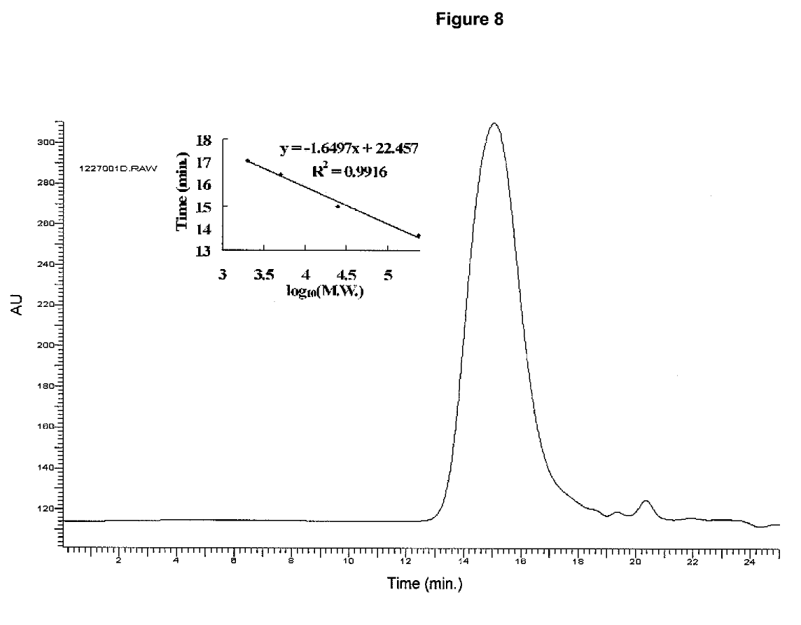

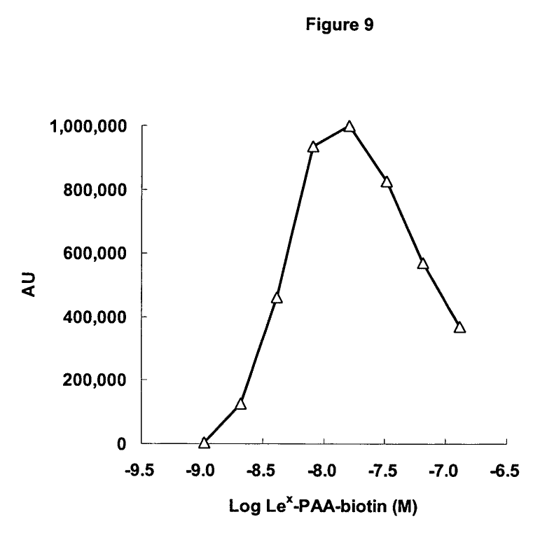

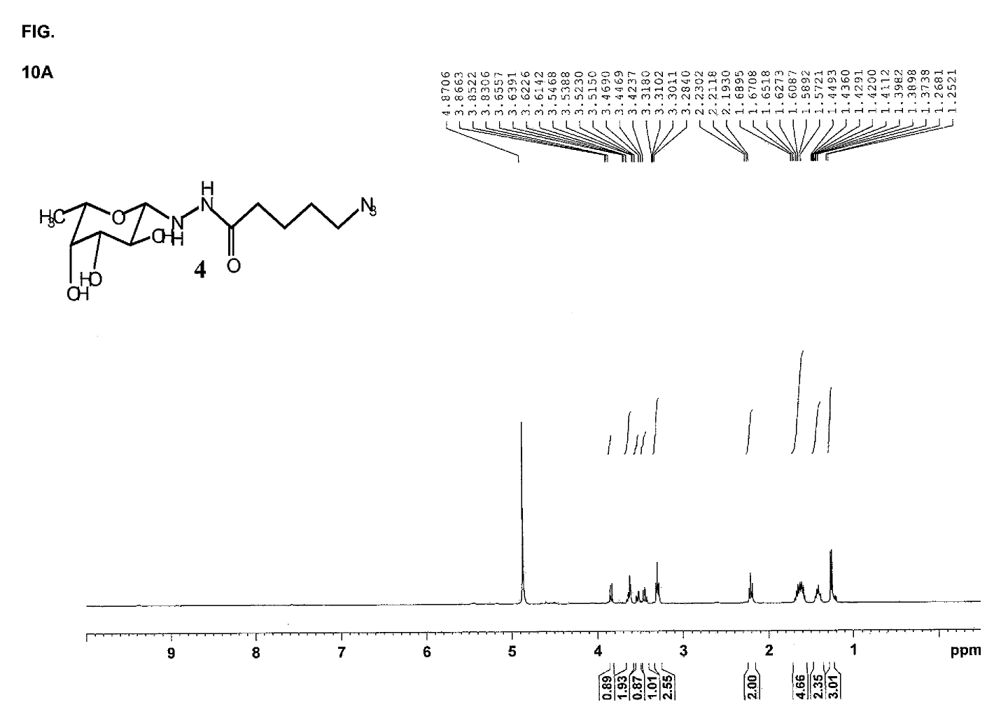

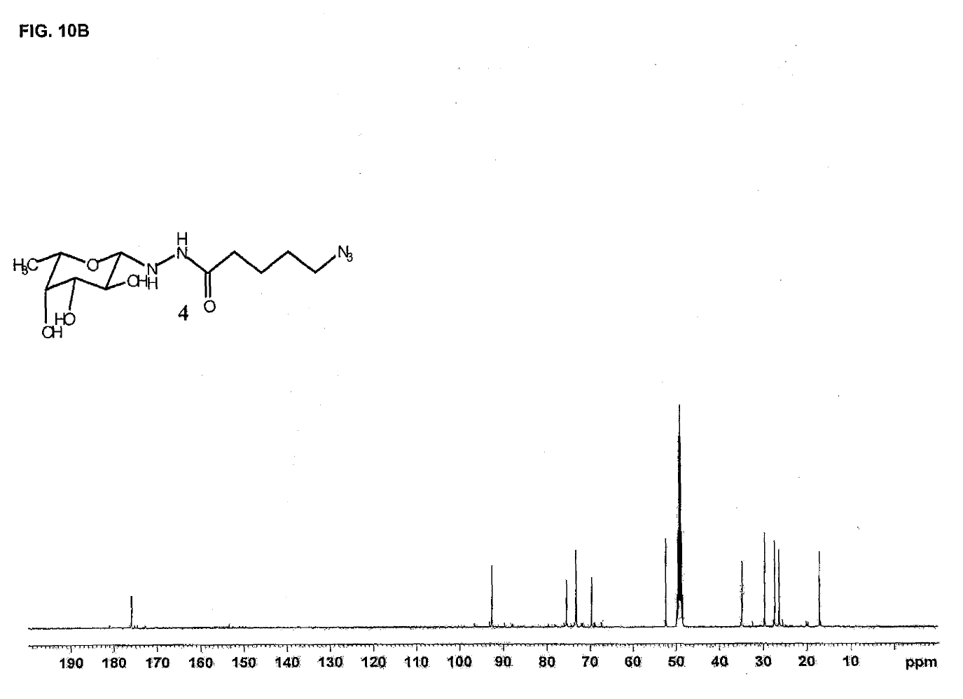

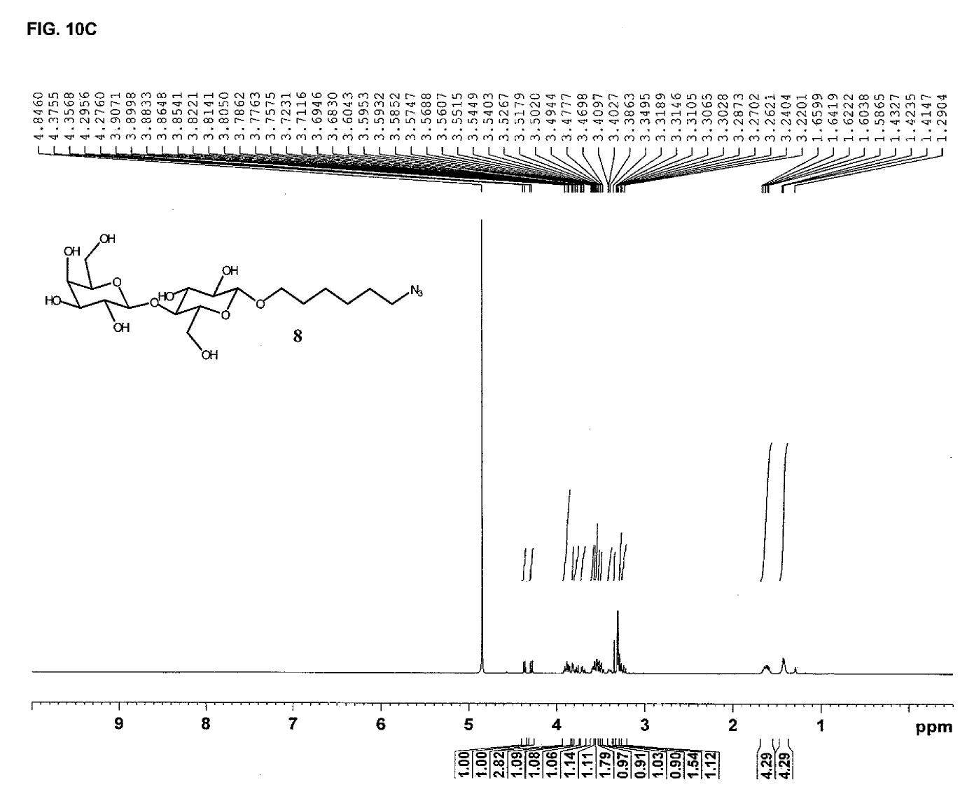

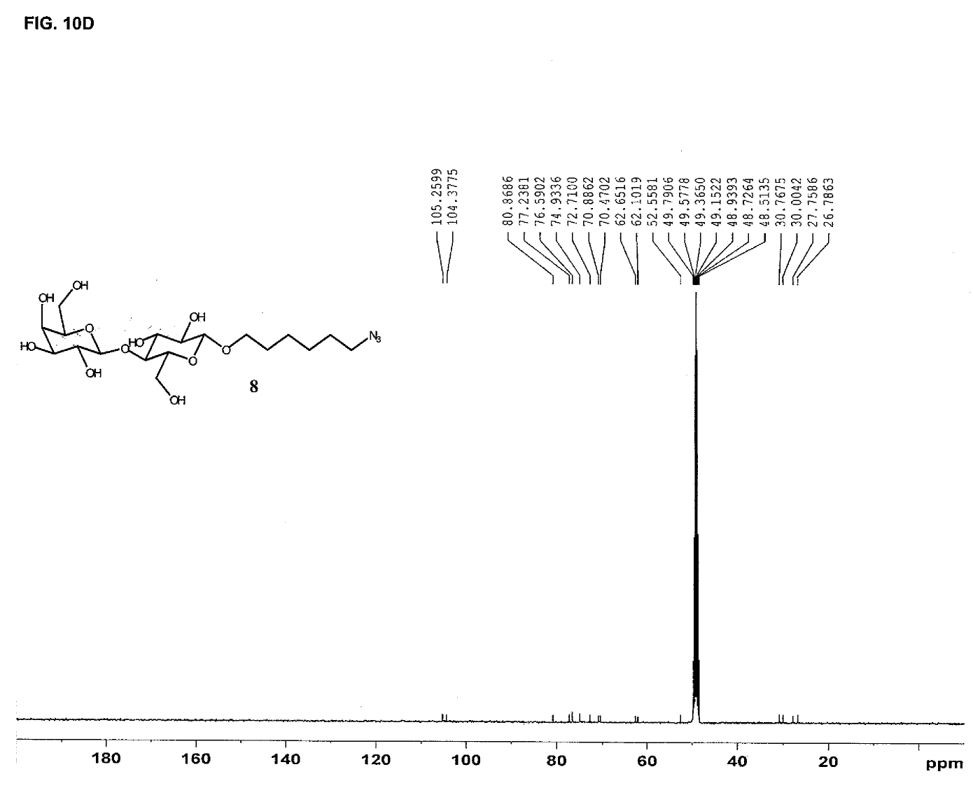

The conclusive results prompted us to profile the receptor specificity of hemagglutinins (HAs) of influenza viruses from various sources. Different recombinant HAs or influenza virus particles were attached to the acceptor beads via the binding of the anti-HA antibodies and then examined with our collection of PAA-sugars (FIGS. 4 and 5). The results of FIGS. 5A and 4A indicate that H1N1 virus (A/Beijing/262/95) and the corresponding HA, respectively, produce a consistent pattern from twelve sialylated sugars. Most of them are a2-3 sialylated to Gal and a2-6 sialylated to GalNAc. The HA or H1N1 virus do not recognize NeuGca2-6GalNAc, NeuAca2-3GalNAc and NeuAca2-8 oligosialic acid.

In contrast, the HA of H3N2 virus (A/Panama/2007/99, FIG. 5B) accepted most of sialic acid-containing sugars including a2-6 sialylated sugars, a2-8 oligosialic acid, and NeuGc-containing sugars. Other H1N1 and H3N2 virus strains also showed similar binding patterns but with different intensities (FIGS. 5C and 5D).

Furthermore, PAA-sugars can exhibit broad diversity because they can be easily derivatized to expand our saccharide reperoires. Enzyme-catalyzed glycosylation is considered as a good approach because it yields exclusive stereoselectivity without the need for additional protection/deprotection steps. For example, 3′HSO3-Galb1,4GlcNAc (No. 19 in Table 1), 3′HSO3-Galb1,3GlcNAc (No. 23) and 3′HSO3-Galb1,3(Fuca1,4)GlcNAc (No. 41) were subjected to sialylation at C6-OH of Gal by Photobacterium damsela a2,6-sialylatransferase[28] (data not shown). It is intriguing that these sialylated products can be recognized by influenza virus H3N2 (A/Panama/2007/99).

In summary, our studies have demonstrated a reproducible and rapid method for characterizing sugar-protein interactions with high sensitivity and minimized materials (in the range of ng per well). All the procedures are carried out in 384-welled microtiter plates, thus qualifying the protocol as high-throughput. This assay carried out in homogeneous solutions prevents the loss of weak bindings which may occur in the repeating washes of sugar microarrays. This method should complement with other array methods using different surfaces.

Experimental Section

General: ALPHAScreen™ assays were carried out on a PerkinElmer Envision instrument. Streptavidin coated donor beads, protein A conjugated acceptor beads and ProxiPlate-384 assay plates were purchased from Perkin Elmer Life Sciences, Inc. (Boston, Mass., U.S.A.). Lectins including Canavalia ensiformis (Con A), Dolichos biflorus (DBA), Maackia amurensis (MAA), Arachis hypogaea (PNA), Glycine max (SBA), Ulex europaeus (UEA-1), Wisteria floribunda (WFA), Triticum vulgaris (WGA), Erythrina cristagalli (ECA), Griffonia simplicifolia I (GS-I) and related rabbit anti-lectin antibodies were purchased from EY Labs, Inc. (San Mateo, Calif., U.S.A.). Lectins of Maackia amurensis (MAL II) and Sambucus Nigra (SNA) were purchased from Vertor Laboratory Inc. (Burlingame, Calif., U.S.A.). Rabbit anti-mouse IgG, Rabbit anti-mouse IgM and Rabbit anti-FITC antibodies were purchased from Zymed, Inc. (South San Francisco, Calif., U.S.A.). Mouse Anti-CD 15 antibody was purchased from BioLegend, Inc. (San Diego, Calif., U.S.A.). Mouse anti-Lea and mouse anti-sialyl Lea antibodies were purchased from Biomeda, Inc. (Foster city, Calif., U.S.A.). Mouse anti-Leb, mouse anti-Ley, mouse anti-6X His tag and mouse anti-influenza virus A antibodies were purchased from Abcam, Ltd. (Cambridge, UK). Mouse anti-sialyl Lex antibody was purchased from Chemicon International Inc. (Temecula, Calif., U.S.A.). Mouse antibodies of anti-blood group type A, B and H were purchased from Acris Antibodies GmbH, (Hiddenhausen, Del.). Biotinylated bovine albumin, human albumin and transferrin were purchased from Rockland Immunological, Inc. (Gilbertsvile, Pa., U.S.A.). Biotinylated poly-acrylamide based sugar polymers were purchased from GlycoTech, (Gaithersburg, Md., U.S.A.). Reagents of the highest purity were purchased from Aldrich, Sigma, Acros, and Novabiochem.

A general procedure for the ALPHAScreen™ assay is shown as follows. All the procedures and incubations must be carried out in the dark. All of the concentrations listed below are final concentrations. Biotin-PAA-sugars (800 pg/mL), lectins (0.5-2.0 ng/mL), glycoproteins (2.0 ng/mL), virus particles (5.3 ng/mL), antibodies (200-500 pg/mL), recombinant hemagglutinin (1.0 ng/mL), donor and acceptor beads (20 ng/mL) were diluted with the assay buffer (50 mM HEPES at pH 7.5, containing 50 mM EDTA and 0.1% BSA w/w) to an appropriate concentration. The anti-lectin or anti-sugar antibodies (1.0 ng/mL), 2nd antibody (1.0-2.0 ng/mL) and acceptor beads (20 ng/mL) were incubated (as the acceptor mixture) in the assay buffer for 1 h at 25° C. before use. Biotin-PAA-sugars or biotin-conjugated glycoproteins, lectins/HAs and donor beads (total 15 mL) were added into the wells of ProxiPlate-384 assay plates separately and incubated at 25° C. for 1 h. An aliquot of the acceptor mixture (10 mL) was then added into the wells (final volume: 50 mL) and incubated at 25° C. for another 2 h. The results were obtained on the PerkinElmer Envision instrument using ALPHAScreen™ program.

The biotinylated polyacrylamide glycoconjugates was synthesized as described by G. M. Whitesides[22] with some modifications. For instance, please see Supporting Information for the details regarding to the preparation of biotinylated PAA-L-fucose (glycan No. 6 in Table 1) and biotinylated PAA-lactose (glycan No. 12 in Table 1).

### PART II

### Preparation of Poly N-acryloxysuccinimide 2

Acryloyl chloride (33.4 g, 30 mL, 369 mmol) was added dropwise to a stirred solution of N-hydroxysuccinimide (42.5 g, 369 mmol) and triethylamine (41.0 g, 56.5 mL, 409 mmol, 1.1 equiv) in CHCl₃ (300 mL, 1.23 M) at 0° C. The solution was allowed to stir for 3 h at 0° C., washed with water (2×300 mL) and brine (300 mL), dried over MgSO₄, and recrystalized from a solution of ethyl acetate/hexane (1:1) to give 46.1 g (273 mmol) colorless crystals of 1 in 72% yield. A mixture of compound 1 (3.15 g, 18.6 mmol) and AIBN (20 mg, 0.007 equiv) in benzene (150 mL) was subjected to polymerization by heating the reaction mixture at 60° C. for 24 h. After the solution was cooled down to 25° C., a white precipitate formed. This precipitate was filtered, washed four times with THF (30 mL), and dried in vacuo to yield 2 (3.08 g, 18.2 mmol, 98%) as a white fluffy solid. The polymer was taken up in dry THF (300 mL), vigorously stirred for 3 days, filtered, and dried in vacuo.

### Determination of the Molecular Weight of 2

The molecular weight distribution of the prepared acrylamide polymers was determined by gel filtration chromatography. The polymer was first hydrolyzed to polyacrylic acid under acidic condition. A solution of 2 (2 mg) in 6 N HC1 (aq, 1 mL) was heated at 105° C. for 24 h in a sealed tube. After being cooled down to 25° C., the solution pH was adjusted to 7.2 with 1 N NaOH, followed by the addition of water (1 mL). The solution was exhaustively dialyzed against phosphate buffer (pH 7.2) that was composed of 150 mM Na₂SO₄ and 10 mM Na₂HPO₄. The resulting sample was analyzed by gel filtration using HPLC (7.8×300 mm column, Waters Ultrahydrogel Linear). The following standards were used for the purpose of calibration, including polyacrylamides (PAA) of MW 5 kD, 25 kD, 100 kD, 200 kD, 1000 kD and 2000 kD.

### Preparation of Compound 5

A solution of 3 (100 mg, 0.54 mmol) and L-fucose (48 mg, 0.87 mmol) in EtOH (5 ml) was stirred at room temperature for 24 h. The solvent was then removed and chromatographed by silica gel chromatography with CHCl₃/MeOH (6:1) to give the coupling product 4 (132 mg, 77% yield). Subsequent reduction of compound 4 was carried out by using triphenylphosphine in THF/H₂O to give the termial amine 5 (129 mg, 90%).

### Preparation of Compound 9

A solution of lactose octaacetate 6 (100 mg, 0.15 mmol) and 1-azido pentanol 7 (50 mg, 0.35 mmol) in anhydrous CH₂Cl₂ (5 ml) was stirred at room temperature under Ar atmosphere, followed by the dropwise addition of BF₃·OEt₂ (0.2 ml, 0.2 mmol). The resulting solution was stirred at room temperature under Ar atmosphere. After 2 h, the reaction was quenched by NaHCO_{3(aq)} and extracted with EtOAc for three times. The collected organic layer was washed with brine, dried over MgSO₄, concentrated to give a dried residue which was further chromatographed by silica gel chromatography with EtOAc/hexane (1:1) to give the desired product 8 (47 mg, 41% yield). Compound 8 dissolved in MeOH was treated with NaOMe and stirred for 6 h to give the deprotected dissacharide 9 (42 mg, 90% yield).

### Preparation of PAA-linked Sugars 10 and 11

A solution of 5 or 9 (243 μmol) in triethylamine (0.5 mL) and biocytin (61 μM) were added to a stirred solution of succinimide ester 2 (6.78 g, 1.22 mmol) in DMF (20 mL). The resulting mixture was stirred at room temperature for 20 h, heated at 65° C. for 6 h, and then stirred at 25° C. for additional 48 h. After dropwise addition of 1.5 mL NH₄OH, the resulting mixture was stirred at room temperature for 12 h, and dialyzed against phosphate buffer saline (pH 7.4) to give PAA-linked sugars 10 or 11.

TABLE 2
(SI). Binding specificities for examined lectins.

| Lectins | Binding specificities |
|---|---|
| Con A | Man, Glc, GlcNAc |
| DBA | GalNAc |
| PNA | Galβ1-3GalNAc |
| SBA | GalNAc/Gal |
| UEA-1 | Fuc |
| WFA | GalNAc |
| WGA | GlcNAcβ1-4GlcNAc, Neu5Ac |
| ECA | Galβ1-4GlcNAc |
| MMA | Gal |
| GS-I | Gal |
| SNA | Neu5Acα2-6 |

### References

[1] C.-H. Wong, J. Org. Chem. 2005, 70, 4219-4225; b) O. Blixt, S. Head, T. Mondala, C. Scanlan, M. E. Huflejt, R. Alvarez, M. C. Bryan, F. Fazio, D. Calarese, J. Stevens, N. Razi, D. J. Stevens, J. J. Skehel, I. van Die, D. R. Burton, I. A. Wilson, R. Cummings, N. Bovin, C.-H. Wong, J. C. Paulson, Proc. Natl. Acad. Sci. U.S.A. 2004, 101, 17033-17038.

[2] A. Varki, Glycobiol. 1993, 3, 97-130; b) M. Fukuda, O. Hindsgaul, Molecular and Cellular Glycobiology, Oxford University Press, Oxford, 2000, pp. 31-43; c) P. Sears, C.-H. Wong, Cell. Mol. Life Sci. 1998, 54, 223-252.

[3] T. Feizi, Carbohydr. Chem. Biol. 2000, 4, 851-860; J. C. Saccheftini, L. G. Baum, C. F. Brewer, Biochemistry 2001, 40, 3009-3015; c) G. Kansas, Blood 1996, 88, 3259-3287; d) T. Geijtenbeck, R. Torensama, S. van Vliet, G. van Duijnhoven, G. Adema, Y. van Kooyk, C. Figdor. Cell 2000, 100, 575-585; e) K. A. Karlsson, Biochem. Soc. Trans. 1999, 27, 471-474.

[4] J. L. Magnani, M. Brockhaus, D. F. Smith, V. Ginsburg, M. Blaszczyk, K. F. Mitchell, Z. Steplewski, H. Koprowski, Science 1981, 212, 55-56; b) J. L. Magnani, B. Nilsson, M. Brockhaus, D. Zopf, Z. Steplewski, H. Koprowski, V. Ginsburg, J. Biol. Chem. 1982, 257,14365-14369; c) K.-E. Falk, K.-A. Karlsson, G. Larson, J. Thurin, M. Blaszczyk, Z. Steplewski, H. Koprowski, Biochem. Biophys. Res. Commun. 1983, 110, 383-391.

[5] J. J. Skehel, D. C. Wiley, Annu. Rev. Biochem. 2000, 69, 531-569.

[6] D. M. Ratner, E. W. Adams, M. D. Disney, P. H. Seeberger, ChemBioChem 2004, 5,1375-1383; b) R. Raman, S. Raguram, G. Venkataraman, J. C. Paulson, R. Sasisekharan, Nature Methods 2005, 2, 817-824.

[7] A. M. Wu, The Molecular Immunology of Complex Carbohydreats-2, Kluwer Academic/Plenum Publishers, 2001, pp.127-132.

[8] E. A. Smith, W. D. Thomas, L. L. Kiessling, R. M. Corn, J. Am. Chem. Soc. 2003, 125, 6140-6148; b) P. A. van der Merwe, A. N. Barclay, Curr. Opin. Immunol. 1996, 8, 257-261; c) M. Vila-Perelló, R. G. Gallego, D. Andreu, ChemBioChem 2005,6,1831-1838; d) D. M. Ratner, E. W. Adams, J. Su, B. R. O'Keefe, M. Mrksich, P. H. Seeberger, ChemBioChem 2004, 5, 379-382.

[9] M. Liebau, A. Hildebrand, R. H. H. Neubert, Eur. Biophys. J. 2001, 30, 42-52; b) Z. Pei, H. Andweson, T. Aastrup, O. Ramström, Biosensors Bioelectronics 2005, 21, 60-66.

[10] D. Beccati, K. M. Halkes, G. D. Batema, G. Guillena, A. C. de Souza, G. van Koten, J. P. Kamerling, ChemBioChem 2005, 6,1196-1203.

[11] S. Fukui, T. Feizi, C. Galustian, A. M. Lawson, W, Chai, Nature Biotechnol. 2002, 20, 1011-1017; b) M. D. Disney, P. H. Seeberger, Chem. Biol. 2004, 11,1701-1707; c) C.-Y. Huang, D. A. Thayer, A. Y. Chang, M. D. Best, J. Hoffmann, S. Head, C.-H. Wong, Proc. Natl. Acad. Sci. U.S.A. 2006, 103, 15-20; d) T. Feizi, F. Fazio, W. Chai, C.-H. Wong, Curr. Opin. Struc. Biol. 2003, 13, 637-645; e) I. Shin, S. Park, M. Lee, Chem. Eur. J. 2005, 11, 2894-2901.

[12] T. Feizi, R. A. Child, Methods Enzymol. 1994, 242, 205-217; b) T. Feizi, W. Chai, Nature Rev. Mol. Cell. Biol. 2004, 5, 582-588.

[13] F. Fazio, M. C. Bryan, O. Blixt, J. C. Paulson, C.-H. Wong, J. Am. Chem, Soc. 2002, 124, 14397-14402.

[14] M. C. Bryan, F. Fazio, H.-K. Lee, C.-Y. Huang, A. Chang, M. D. Best, D. A. Calarese, O. Blixt, J. C. Paulson, D. Burton, I. A. Wilson, C.-H. Wong, J. Am. Chem. Soc. 2004, 126, 8640-8641.

[15] K.-S. Ko, F. A. Jaipuri, N. L. Pohl, J. Am. Chem. Soc. 2005, 127, 13162-13163.

[16] V. I. Dyukova, E. I. Demantieva, D. A. Zubtsov, O. E. Galanina, N. V. Bovin, A. Y. Rubina, Anal. Biochem. 2005, 347, 94-105.

[17] J. Hirabayashi, Y. Arata, K. Kasai, J. Chromatography A 2000, 890, 261-271; b) B. Zhang, M. M. Palcic, D. C. Schriemer. G. Alvarez-Manilla, M. Pierce, O. Hindsgaul, Anal. Biochem. 2001, 299, 173-182; c) J. Hirabayashi, Glycoconj. J. 2004, 21, 35-40; d) A. Taga, Y. Yamamoto, R. Maruyama, S. Honda, Electrophoresis 2004, 25, 876-881.

[18] A. Kuno, N. Uchiyama, S. Koseki-Kuno, Y. Ebe, S. Takashima, M. Yamada, J. Hirabayashi, Nature Methods 2005, 2, 851-856.

[19] M. I. Khan, N. Surolia, M. K. Mathew, P. Balaram, A. Surölia, Eur. J. Biochem. 1981, 115, 149-152; b) Y. C. Lee, J. Biochem. 1997, 121, 818-825; c) P. Sörme, B. Kahl-Knutsson, M. Huflejt, U. J. Nilsson, H. Leffler, Anal. Biochem. 2004, 334, 36-47

[20] W. H. Pohl, H. Hellmuth, M. Hilbert, J. Seibel, P. J. Walla, ChemBioChem 2006, 7, 268-274.

[21] J. Su, M. Mrksich, Angew. Chem. Int. Ed. 2002, 41, 4715-4718.

[22] C. I. Gama, S. E. Tully, N. Sotogaku, P. M. Clark, M. Rawat, N. Vaidehi, W. A. Goddard III, A. Nishi, L. C. Hsieh-Wilson, Nature Chem. Biol. 2006, 2, 467-473.

[23] E. L. Shipp, L. C. Hsieh-Wilson, Chem. Biol. 2007, 14, 195-208.

[24] E. F. Ullman, H. Kirakossian, S. Singh, Z. P. Wu, B. R. Irvin, J. S. Pease, A. C. Switchenko, J. D. Irvine, A. Dafforn, C. N. Skold, Proc. Natl. Acad. Sci. U.S.A. 1994, 91, 5426-5430; b) E. F. Ullman, H. Kirakossian, A. C. Switchenko, J. Ishkanian, M. Ericson, C. A. Wartchow, M. Pirio, J. Pease, B. R. Irvin, S. Singh, Clin. Chem., 1996, 42, 1518-1526; c) R. Bossé, C. Illy, J. Elands, D. Chelsky, Drug Discovery Today, 2000, 5 (Supplement 1), 42-47.

[25] M. A. Sparks, K. W. Williams, G. M. Whitesides, J. Med. Chem. 1993, 36, 778-783; b) W. J. Lees, A. Spaltenstein, J. E. Kingery-Wood, G. M. Whitesides, J. Med. Chem. 1994, 37, 3419-3433; c) M. Mammen, G. Dahmann, G. M. Whitesides, J. Med. Chem. 1995, 38, 4179-4190; d) G. B. Sigal, M. Mammen, G. Dahmann, G. M. Whitesides, J. Am. Chem. Soc. 1996, 118. 3789-3800.

[26] D. Koirrich, F. Altmann, Anal. Biochem. 2000, 285, 64-75; b) K. T. Pilobello, L. Krishnamoorthy, D. Slawek, L. K. Mahal, ChemBioChem 2005, 6, 985-989; c) L. Yang, Q. Tang, A. K. Harrata, C. S. Lee, Anal. Biochem. 1996, 243, 140-149; d) N. G. Karlsson, N. H. Packer, Anal. Biochem. 2002, 305, 173-185.

[27] S. Angeloni, J. L. Ridet, N. Kusy, H. Gao, F. Crevoisier, S. Guinchard, S. Kochhar, H. Sigrist, N. Sprenger, Glycobiol. 2005, 15, 31-41.

[28] C.-F. Teo, T.-S. Hwang, P.-H. Chen, C.-H. Hung, H.-S. Gao, L.-S. Chang, C.-H. Lin, Adv. Synth. Catal. 2005, 347, 967-972.

1. A donor bead for use in an assay, wherein the bead (a) is coated with a layer of hydrogel having directly or indirectly bound thereto a polyacrylamide-supported sugar or a polyacrylamide-supported glycan, and (b) comprises a photosensitizer which, upon excitation by laser light of a suitable wavelength, converts ambient oxygen to singlet state oxygen.

2. The donor bead of claim 1, wherein (a) the bead is coated with streptavidin and the polyacrylamide-supported sugar or polyacrylamide-supported glycan is bound to the bead via a biotin/streptavidin link, and (b) the photosensitizer is pthalocyanine.

3. The donor bead of claim 1, wherein the bead has a polyacrylamide-supported glycan bound thereto.

4. An acceptor bead for use in an assay, wherein the bead (a) is coated with a layer of hydrogel having directly or indirectly bound thereto a polyacrylamide-supported sugar or a polyacrylamide-supported glycan, and (b) comprises a chemiluminescer and a fluorophore, whereby when the bead is contacted with singlet state oxygen, the singlet state oxygen reacts with the chemiluminescer which in turn activates the fluorophore so as to cause the emission of light of a predetermined wavelength.

5. The acceptor bead of claim 4, wherein (a) the bead is coated with streptavidin and the polyacrylamide-supported sugar or a polyacrylamide-supported glycan is bound to the bead via a biotin/streptavidin link, (b) the chemiluminescer is a thioxene derivative which luminesces at a wavelength of 370 nm, and (c) the fluorophore shifts 370 nm luminescence to a wavelength of from 520 nm to 620 nm.

6. The donor bead of claim 4, wherein the bead has a polyacrylamide-supported glycan bound thereto.

7. A kit comprising, in separate compartments, (a) a donor bead (i) coated with a layer of hydrogel and (ii) comprising a photosensitizer which, upon excitation by laser light of a suitable wavelength, converts ambient oxygen to singlet state oxygen, and (b) (i) a polyacrylamide-supported sugar or a polyacrylamide-supported glycan, or (ii) reagents for making a polyacrylamide-supported sugar or a polyacrylamide-supported glycan.

8. The kit of claim 7, wherein (a) the donor bead is further coated with streptavidin, (b) the polyacrylamide-supported sugar or polyacrylamide-supported glycan is conjugated with biotin, and (c) the photosensitizer is pthalocyanine.

9. The kit of claim 7 further comprising, in a separate compartment, an acceptor bead comprising a chemiluminescer and a fluorophore.

10. The kit of claim 9, wherein the chemiluminescer is a thioxene derivative which luminesces at a wavelength of 370 nm, and the fluorophore shifts 370 nm luminescence to a wavelength of from 520 nm to 620 nm.

11. A kit comprising, in separate compartments, (a) an acceptor bead (i) coated with a layer of hydrogel and (ii) comprising a chemiluminescer and a fluorophore, whereby when the bead is contacted with singlet state oxygen, the singlet state oxygen reacts with the chemiluminescer which in turn activates the fluorophore so as to cause the emission of light of a predetermined wavelength, and (b) (i) a polyacrylamide-supported sugar or a polyacrylamide-supported glycan, or (ii) reagents for making a polyacrylamide-supported sugar or a polyacrylamide-supported glycan.

12. The kit of claim 11, wherein (a) the acceptor bead is further coated with streptavidin, (b) the polyacrylamide-supported sugar or polyacrylamide-supported glycan is conjugated with biotin, (c) the chemiluminescer is a thioxene derivative which luminesces at a wavelength of 370 nm, and (d) the fluorophore shifts 370 nm luminescence to a wavelength of from 520 nm to 620 nm.

13. The kit of claim 11 further comprising, in a separate compartment, a donor bead comprising a photosensitizer.

14. The kit of claim 13, wherein the photosensitizer is pthalocyanine.

15. A method for determining whether a lectin or antibody binds to a sugar or glycan comprising (a) contacting, under binding-permitting conditions, (i) the donor bead of claim 1 having bound to its surface the sugar or glycan in polyacrylamide-supported form, and (ii) an acceptor bead having the lectin or antibody bound to its surface, wherein the acceptor bead comprises a chemiluminescer and a fluorophore, whereby when the acceptor bead is contacted with singlet state oxygen, the singlet state oxygen reacts with the chemiluminescer which in turn activates the fluorophore so as to cause the emission of light of a predetermined wavelength, (b) exposing the resulting beads to laser light of a wavelength which excites the photosensitizer in the donor bead, and (c) determining whether light is emitted by the fluorophore in the acceptor bead, the emission of light indicating that the lectin or antibody binds to the sugar or glycan.

16. The method of claim 15, wherein (a) the donor bead is coated with streptavidin and the polyacrylamide-supported sugar or polyacrylamide-supported glycan is bound to the bead via a biotin/streptavidin link, (b) the photosensitizer is pthalocyanine, (c) the acceptor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm.

17. The method of claim 15, wherein the donor bead has a polyacrylamide-supported glycan bound thereto.

18. The method of claim 15, wherein the method is performed using an assay well plate.

19. A method for determining whether a lectin or antibody binds to a sugar or glycan comprising (a) contacting, under binding-permitting conditions, (i) a donor bead having the lectin or antibody bound to its surface, and (ii) the acceptor bead of claim 4 having bound to its surface the sugar in polyacrylamide-supported form or the glycan in polyacrylamide-supported form, wherein the donor bead comprises a photosensitizer which, upon excitation by laser light of a suitable wavelength, converts ambient oxygen to singlet state oxygen, (b) exposing the resulting beads to laser light of a wavelength which excites the photosensitizer in the donor bead, and (c) determining whether light is emitted by the fluorophore in the acceptor bead, the emission of light indicating that the lectin or antibody binds to the sugar or glycan.

20. The method of claim 19, wherein (a) the acceptor bead is coated with streptavidin and the polyacrylamide-supported sugar or polyacrylamide-supported glycan is bound to the bead via a biotin/streptavidin link, (b) the photosensitizer is pthalocyanine, (c) the donor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm.

21. The method of claim 19, wherein the acceptor bead has a polyacrylamide-supported glycan bound thereto.

22. The method of claim 19, wherein the method is performed using an assay well plate.

23. A method for characterizing a glycan with respect to the makeup of its sugar moieties comprising (a) contacting, under binding-permitting conditions, (i) donor beads of claim 1, each having the glycan bound to its surface in polyacrylamide-supported form, and (ii) a plurality of acceptor beads, each having bound to its surface a lectin or antibody recognizing a predetermined sugar moiety, wherein the acceptor bead comprises a chemiluminescer and a fluorophore, whereby when the acceptor bead is contacted with singlet state oxygen, the singlet state oxygen reacts with the chemiluminescer which in turn activates the fluorophore so as to cause the emission of light of a predetermined wavelength, and wherein acceptor beads having a lectin or antibody recognizing a predetermined sugar moiety are contacted with the donor beads in a compartment separate from those in which donor beads are contacted with acceptor beads having lectins or antibodies recognizing other predetermined sugar moieties, (b) exposing the resulting beads in each compartment to laser light of a wavelength which excites the photosensitizer in the donor beads, and (c) for each compartment, determining whether light is emitted by the fluorophore in the respective acceptor beads and thus whether the lectin or antibody is bound to its respective sugar moiety on the glycan, thereby characterizing the glycan.

24. The method of claim 23, wherein (a) the donor bead is coated with streptavidin and the polyacrylamide-supported glycan is bound to the bead via a biotin/streptavidin link, (b) the photosensitizer is pthalocyanine, (c) the acceptor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm.

25. The method of claim 23, wherein the method is performed using an assay well plate.

26. A method for characterizing a glycan with respect to the makeup of its sugar moieties comprising (a) contacting, under binding-permitting conditions, (i) acceptor beads of claim 4, each having the glycan bound to its surface in polyacrylamide-supported form, and (ii) a plurality of donor beads, each having bound to its surface a lectin or antibody recognizing a predetermined sugar moiety, wherein each donor bead comprises a photosensitizer which, upon excitation by laser light of a suitable wavelength, converts ambient oxygen to singlet state oxygen, and wherein donor beads having a lectin or antibody recognizing a predetermined sugar moiety are contacted with the acceptor beads in a compartment separate from those in which acceptor beads are contacted with donor beads having lectins or antibodies recognizing other predetermined sugar moieties, (b) exposing the resulting beads in each compartment to laser light of a wavelength which excites the photosensitizer in the donor beads, and (c) for each compartment, determining whether light is emitted by the fluorophore in the respective acceptor beads and thus whether the lectin or antibody is bound to its respective sugar moiety on the glycan, thereby characterizing the glycan.

27. The method of claim 26, wherein (a) the acceptor bead is coated with streptavidin and the polyacrylamide-supported glycan is bound to the bead via a biotin/streptavidin link, (b) the photosensitizer is pthalocyanine, (c) the donor bead is coated with protein A to which the lectin or antibody is bound, (d) the chemiluminescer is a thioxene derivative which luminesces at a wavelength of 370 nm, (e) the fluorophore shifts 370 nm luminescence to a wavelength of from 520 nm to 620 nm, and (f) the laser light to which the beads are exposed is at a wavelength of 680 nm.

28. The method of claim 26, wherein the method is performed using an assay well plate.
